(19) United States
(12) Patent Application Publication
(10) Pub. No.: US 2013/0125899 A1
(43) Pub. Date: May 23, 2013

(54) MINIMALLY INVASIVE DELIVERY DEVICES AND METHODS

(71) Applicant: Conceptus, Inc., Mountain View, CA (US)

(72) Inventors: Julian Cruzada, San Jose, CA (US); Betsy Swann, Grass Valley, CA (US)

(73) Assignee: CONCEPTUS, INC., Mountain View, CA (US)

(21) Appl. No.: 13/741,289

(22) Filed: Jan. 14, 2013

Related U.S. Application Data
(62) Division of application No. 12/562,065, filed on Sep. 17, 2009.

(51) Int. Cl. A61F 6/06

(52) U.S. Cl. CPC A61F 6/06
USPC 128/830

(57) ABSTRACT

Embodiments of the present invention provide systems and methods for delivering a contraceptive device to an ovarian pathway. The contraceptive device is within an enclosure in a first position. In a second position, the contraceptive device is positioned at the ovarian pathway, by actuating a first actuator. The contraceptive device is then expanded and released from the delivery system by actuating a second actuator.

### RELATED APPLICATIONS

The present application is a divisional of U.S. patent application Ser. No. 12/562,065 filed on Sep. 17, 2009, which is incorporated herein by reference.

### FIELD

The present invention relates to the field of minimally invasive medical devices and procedures and, in particular, to devices and methods for transcervical gynecological procedures.

### BACKGROUND

Female contraception and/or sterilization may be affected by transcervically introducing an object (e.g. a coil) into a fallopian tube to inhibit conception. Devices, systems and methods for such a contraceptive approach have been described in various patents and patent applications assigned to the present assignee. For example, PCT Patent Application No. 99/15116, U.S. Pat. No. 6,526,979 and U.S. Pat. No. 6,634,361, which are hereby incorporated herein in their entirety, describe devices that are transcervically inserted into an ostium of a fallopian tube and mechanically anchored within the fallopian tube. The devices described in these patents and patent application may promote a tissue in-growth around and within the inserted device, which may be referred to as an implant or an insert. One example of such devices is the device known as “Essure” from Conceptus, Inc. of Mountain View, Calif. This tissue in-growth tends to provide long-term contraception and/or permanent sterilization without the need for surgical procedures.

U.S. Pat. No. 6,709,667 and U.S. Publication No. 2008/0041394, which are hereby incorporated herein in their entirety, and which are assigned to the present assignee, describe delivery systems and methods for these devices. The delivery system is typically formed of a handle, a delivery catheter system, and a guidewire onto which is held the contraceptive implant to be placed within the fallopian tube. The delivery catheter system contains the guidewire, a release catheter and the contraceptive implant and the guidewire within the release catheter.

The delivery catheter system is transcervically positioned into the uterus and the fallopian tubes via a hysteroscope. The delivery catheter system and guidewire enter the hysteroscope through a working channel of the hysteroscope. A distention valve is typically positioned at the tip of the working channel. The distention valve seals the entrance of the working channel to prevent a distention fluid, such as saline, to flow out of the hysteroscope as a device, such as the delivery catheter system and guidewire of the intrafallopian contraceptive delivery device, is introduced into the working channel. The opening into the distention valve is designed to prevent the leakage of any fluid out of the hysteroscope and therefore has the smallest opening possible to allow a very tight fit between the device and the valve opening. To prevent damaging the tip of the guidewire or the contraceptive implant to be inserted into the fallopian tube, the guidewire and delivery catheter system are introduced into the distention valve through an introducer sheath. The introducer sheath is formed of a soft flexible material such as plastic or Teflon and has a slit to aid in grasping and in the removal of the introducer sheath. The introducer sheath must therefore be inserted into the opening of the distention valve while on a stiff mandrel. Once the mandrel is placed within the distention valve and the channel to the desired depth the mandrel is removed, leaving the introducer sheath within the working channel and the distention valve. After placing the introducer sheath into the distention valve the tip of the guidewire and the delivery catheter system may be inserted into the introducer sheath and introduced into the distention valve and the working channel. The introducer sheath may then be removed. The distention valve may have a tight opening that places pressure on the delivery catheter and causes friction. The distention valve prevents fluid leakage from the working channel. When the introducer sheath is inserted through the distention valve, fluid can spray out of the valve and onto the physician or physician's assistant. The amount of fluid spray-back can be significant depending on the fluid pressure during the procedure. In addition, friction between the introducer sheath and distention valve can be problematic.

Once a physician has positioned the delivery catheter system and the guidewire at a position within the fallopian tube where the contraceptive implant may be deposited, it may be awkward and difficult for the physician to maintain the position and may require the physician to use an assistant to aid in the proper stabilization of the system relative to the hysteroscope. In addition, some of the contraceptive implant devices in the above references require disengaging from a delivery catheter by using an axial torque. In practice this requires the delivery catheter and endoscope in combination to be fully rotated to disengage a contraceptive implant device from the delivery catheter in order to deposit the contraceptive implant device into a fallopian tube. This maneuver may be difficult and cumbersome to perform considering that the device must also remain axially aligned in the fallopian tube.

A stabilization device may be used to help with this awkwardness. One such stabilization device includes a handle that includes a contoured holster to couple the hysteroscope and the handle of the control device, creating a fixed distance between the hysteroscope and the control device. The stabilization device may also be coupled to an endoscope.

Once the delivery catheter system is positioned at the fallopian tubes, the implant is positioned in the fallopian tube, expanded, and released from the delivery catheter system by rotating a wheel on the handle. One problem with this wheel is that the wheel sometimes sticks causing the doctor to look away from the hysteroscope.

### SUMMARY

Various different embodiments are disclosed below and the following summary provides a brief description of only some of these embodiments. According to one aspect of the invention, certain embodiments described below relate to a medical device that provides a transcervical pathway and also stabilizes a device for a minimally invasive gynecological procedure. The device for the minimally invasive gynecological procedure may be an intrafallopian contraceptive delivery device.

One embodiment of the present invention relates to an implant delivery device having a housing; an expandable implant; a release catheter, the expandable implant releasably coupled with the release catheter; a delivery catheter, the release catheter and the expandable implant slideable through the delivery catheter; a spool in the housing, at least a portion of the delivery catheter wound around the spool in a first position; a first actuator coupled with the housing and the spool to rotate the spool to unwind the delivery catheter from the spool in a second position; and a second actuator coupled with the housing and the spool to release the implant from the release catheter.

Another embodiment of the present invention relates to a system for delivering an implant to an ovarian pathway of a female body including a delivery sheath having a first longitudinal opening and a second longitudinal opening; a visualization system deliverable through the first longitudinal opening; and an implant delivery device deliverable through the second longitudinal device, the implant delivery device having an enclosure, a delivery catheter within the enclosure in a first position, at least a portion of the delivery catheter extending out of the enclosure through the second longitudinal opening in a second position.

In a further embodiment, the present invention relates to a method for delivering a contraceptive device to an ovarian pathway including in response to actuation of a first actuator, unwinding a catheter wound around a spool in an enclosure to deliver the contraceptive device near the ovarian pathway; and in response to actuation of a second actuator, releasing the contraceptive device from the catheter.

Various other devices and methods for using devices, including kits for use in treating patients, are also described below. Other features of the present invention will be apparent from the accompanying drawings and from the detailed description that follows.

### BRIEF DESCRIPTION OF THE DRAWINGS

The invention is described by way of example with reference to the accompanying drawings, wherein:

FIG. 1 is a schematic view illustrating the uterine and tubal anatomy for deployment of the contraceptive devices;

FIG. 1A is a flow diagram of a method for delivering a contraceptive device in accordance with one embodiment of the invention;

FIG. 2 is a front perspective view of a delivery sheath in accordance with one embodiment of the invention;

FIG. 3 is a rear perspective view of the delivery sheath of FIG. 2 in accordance with one embodiment of the invention;

FIG. 4 is a perspective view of the delivery sheath, device delivery system and hysteroscope in accordance with one embodiment of the invention;

FIG. 5 is a side view of the delivery device system in accordance with one embodiment of the invention;

FIG. 6 is a side sectional view of the delivery device system in accordance with one embodiment of the invention;

FIG. 7 is a side exploded, sectional view of the delivery device system in accordance with one embodiment of the invention;

FIGS. 8A and 8B are side views of a distal end of the device delivery system in accordance with one embodiment of the invention;

FIG. 9 is a side view of the implant in accordance with one embodiment of the invention;

FIG. 10 is a schematic view illustrating delivery of the implant to a fallopian tube with the delivery sheath in accordance with one embodiment of the invention;

FIG. 11 and FIG. 11A illustrate delivery of a contraceptive device in accordance with one embodiment of the invention;

FIG. 12 and FIG. 12A illustrate delivery of a contraceptive device in accordance with one embodiment of the invention;

FIG. 13 and FIG. 13A illustrate delivery of a contraceptive device in accordance with one embodiment of the invention;

FIG. 14 and FIG. 14A illustrate delivery of a contraceptive device in accordance with one embodiment of the invention;

FIG. 15 and FIG. 15A illustrate delivery of a contraceptive device in accordance with one embodiment of the invention;

FIG. 16 is a perspective view of the delivery sheath, a guide, device delivery system and hysteroscope in accordance with one embodiment of the invention;

FIG. 17 is a side view of the guide in accordance with one embodiment of the invention;

FIG. 18 is a top view of the guide in accordance with one embodiment of the invention;

FIG. 19 is a schematic view of an adaptor in accordance with one embodiment of the invention;

FIG. 20 is a front perspective view of an alternative embodiment of the invention;

FIG. 21 is a side sectional view of the delivery device system in accordance with an alternative embodiment of the invention;

FIG. 22 is a side exploded, sectional view of the delivery device system in accordance with an alternative embodiment of the invention;

FIGS. 23A and 23B are side sectional views of the delivery device system, delivery catheter, expandable implant and release catheter in accordance with an alternative embodiment of the invention.

### DETAILED DESCRIPTION

Embodiments of the present invention relate to an implant delivery device and a method of delivering the implant to an ovarian pathway. In one embodiment, the implant delivery system includes a housing, an expandable implant, a release catheter, a delivery catheter and first and second actuators. The expandable implant releasably coupled with the release catheter, and the release catheter and the expandable implant are slideable through the delivery catheter. The housing also includes a spool. In a first position, the delivery catheter is entirely within the housing and at least a portion of the delivery catheter is wound around the spool. Actuation of the first actuator rotates the spool to unwind the delivery catheter from the spool and deliver the distal end of the delivery catheter to the ovarian pathway (in a second position). Actuation of the second actuator expands the implant and releases the implant from the release catheter.

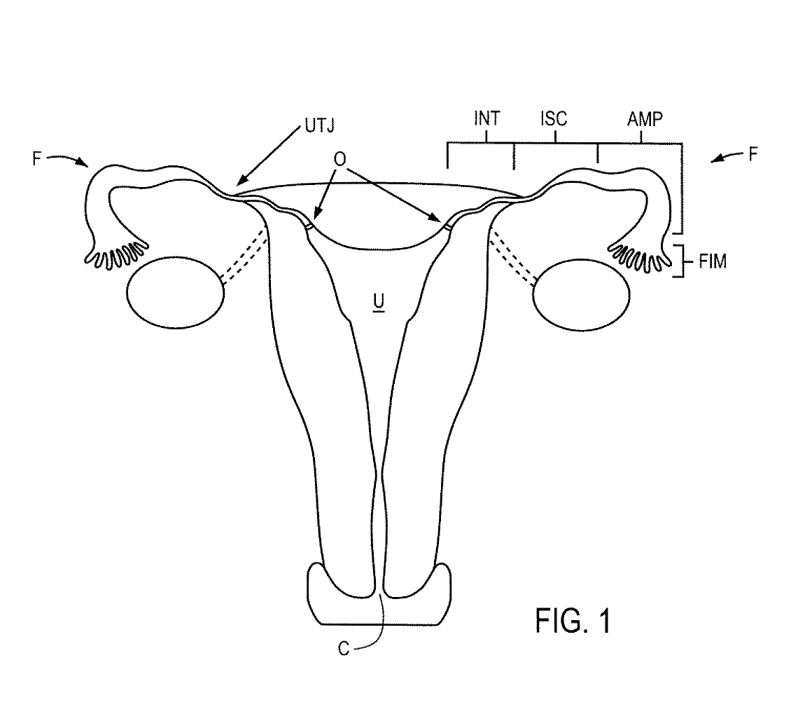

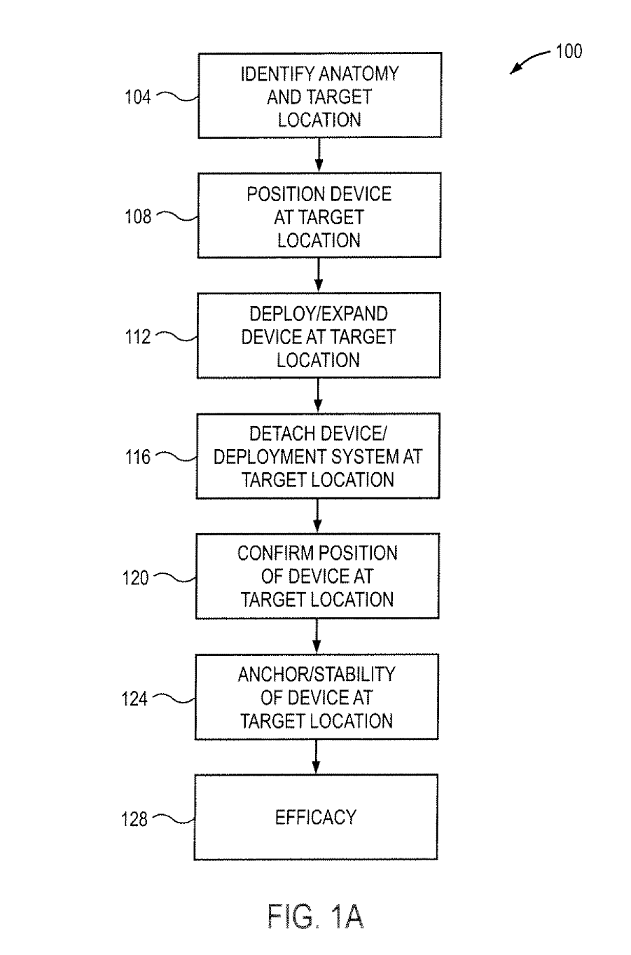

Referring now to FIG. 1, access to uterus U is gained through the cervix C. From within the uterus U, the fallopian tubes F are accessed via the tubal ostiums O. The fallopian tubes F generally include three segments between the ostium O and the fimbria FIM. Beginning adjacent the uterus U, the intramural segment INT of the fallopian tubes F are surrounded by the muscular uterine tissues. Beginning at the uterotubal junction UTJ, the fallopian tubes F extend beyond the uterine tissues and within the peritoneal cavity along an isthmic segment ISC, and then along an ampullary segment AMP.

Referring now to FIG. 1A, an exemplary method 100 for delivering a contraceptive device to a fallopian tube is illustrated. It will be appreciated that the exemplary method 100 may include a fewer or greater number of steps than those described below. In addition, the steps may occur in different orders than described or may occur simultaneously.

The method 100 begins at block 104 by identifying the anatomy and target location. The operator determines a preferred placement of the contraceptive device within the ostium, and also determines if any special circumstances are present for a particular device placement procedure. Anatomy and target location identification can be facilitated using a variety of known visualization modes, including hysteroscopy, sonography (ultrasound), fluoroscopy, and the like.

The method 100 continues at block 108 by positioning the device at the target location. A wide variety of techniques may be used to assist a healthcare professional in positioning the device in the correct location, including visualization techniques, providing high-contrast markers (such as radiopaque markers, echogenic markers, or the like), providing tactile indication of the placement position by including physical stops or “bumpers” (which may be adapted to engage reference tissues in such a tactile way as to send a signal to the healthcare professional), or the like.

The method 100 continues by deploying and/or expanding the device at the target location (block 112), and detaching the device from the deployment system (block 116). The method 100 may continue by confirming the position of the device at the target location (block 120). Confirmation may be provided by visualizing at least a portion of the device after detachment, often using the same visualization modality used during placement. In addition to optical visualization techniques, confirmation of delivery may be provided by including radiopaque markers for fluoroscopic placement confirmation, sonographic markers for ultrasound placement confirmation, or the like.

The method 100 may include anchoring and stabilizing the device at the target location (block 124) and efficacy (block 128). Efficacy may be provided by incorporating a lumen/space filling design in the implant, such as polyester fibers to incite a tissue reaction (i.e., tissue in-growth fibers), that sufficiently alters the function and architecture of the fallopian tube so as to inhibit conception. This tissue reaction results in the incorporation of the contraceptive device into the tubal lumen tissues, so that the device is firmly embedded into the surrounding tissue structure.

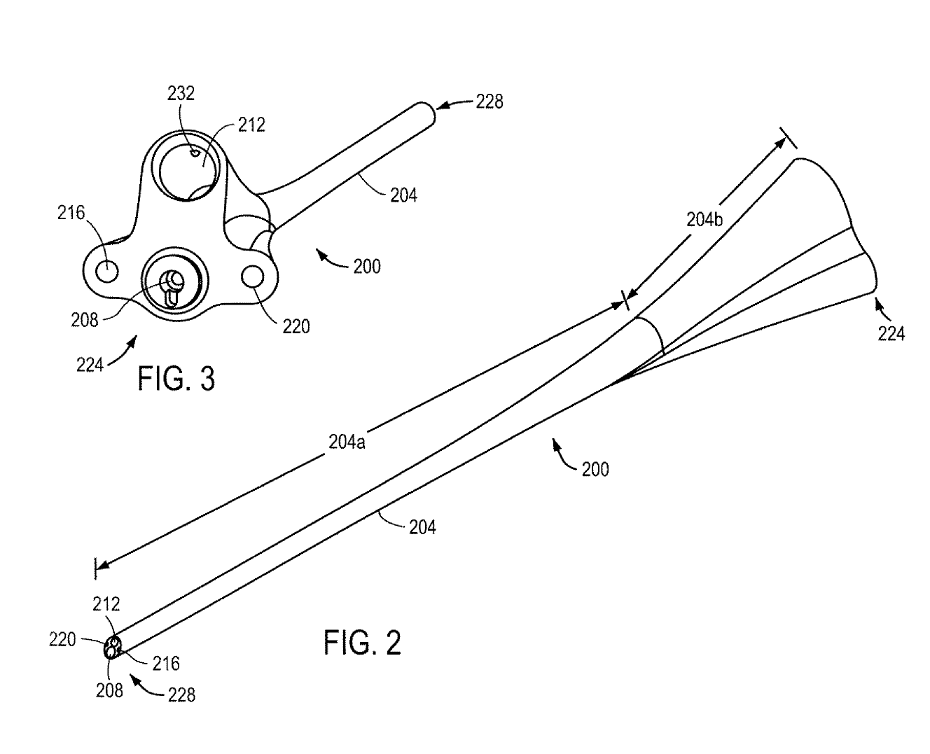

FIGS. 2 and 3 illustrate a delivery sheath 200. The delivery sheath 200 is configured to deliver a contraceptive device to a fallopian tube through the cervix C and uterus U. The delivery sheath 200 includes an elongate body 204 that includes a first lumen 208 and a second lumen 212. The elongate body 204 may optionally include a third lumen 216 and/or fourth lumen 220.

The elongate body 204 includes a proximal end 224 and a distal end 228. As shown in FIGS. 2 and 3, the proximal end 224 of the elongate body 204 is larger than the distal end 228 of the elongate body 204. The proximal end 224 extends out of the cervix and the patient such that an operator can deliver the contraceptive device through the sheath 200, while the distal end 228 extends to or near the ostium O.

The length of the elongate body 204 is configured to provide a transcervical pathway. For example, the length of the elongate body may be about 29-30 cm. In one embodiment, the elongate body 204 includes a working length 204a that extends through the cervix C and uterus U to or near the ostium O, and a manipulating length 204b that extends away from the cervix C and out of the female patient. In one embodiment, the working length 204a may be about 19-20 cm. It will be appreciated that the length of the elongate body may be less than 29 cm or greater than 30 cm and the working length may be less than 19 cm or greater than 20 cm.

The first lumen 208 extends from the proximal end 224 to the distal end 228 of the elongate body. The first lumen 208 is configured to receive a visualization tool, such as, for example, a hysteroscope. The visualization tool may be secured to the sheath 200 through a locking mechanism, such as, for example, a groove and tab, at a proximal end of the first lumen 208. Alternatively, the visualization tool is not secured to the sheath 200.

The hysteroscope is positioned in the sheath so that an operator can view the fallopian tube sufficiently to deliver the implant in the fallopian tube. In one embodiment, the distal end of the hysteroscope is aligned with the distal end of the sheath 200 (i.e., the distal end of the hysteroscope is flush with the distal end of the sheath 200). In another embodiment, the hysteroscope extends just beyond the distal end 228 of the elongate body 204. For example, the hysteroscope may extend less than about 1 cm beyond the distal end 228 of the elongate body.

The second lumen 212 also extends from the proximal end 224 to the distal end 228 of the elongate body. The second lumen 212 is configured to receive a device delivery system. The device delivery system may be secured to the sheath 200 through a locking mechanism, such as, for example, a groove and tab at a proximal end of the second lumen 212. As shown in FIG. 3, the second lumen 212 includes a tab 232 to interlock the delivery sheath with a corresponding groove in a device delivery system, described in further detail hereinafter.

The first lumen 208, second lumen 212, both the first lumen 208 and second lumen 212 or neither the first lumen 208 nor the second lumen 212 may include a distension valve to prevent fluid leakage through the sheath 200.

The optional third lumen 216 and fourth lumen 220 are configured to be fluid lumens. For example, the third lumen 216 may be a fluid inlet and the fourth lumen 220 may be a fluid outlet, or vice versa. Alternatively, a single lumen (e.g., only third lumen 216) is provided. When a single lumen is provided, the single lumen may be both a fluid inlet and fluid outlet, or the single lumen may be a fluid inlet, while the second lumen 212, for example, serves as the fluid outlet as well as a working channel.

In one embodiment, one or more tendons are connected to the distal end of the sheath 200 to allow the operator to deflect the sheath 200 to direct the distal end of the sheath 200 toward the ostium O.

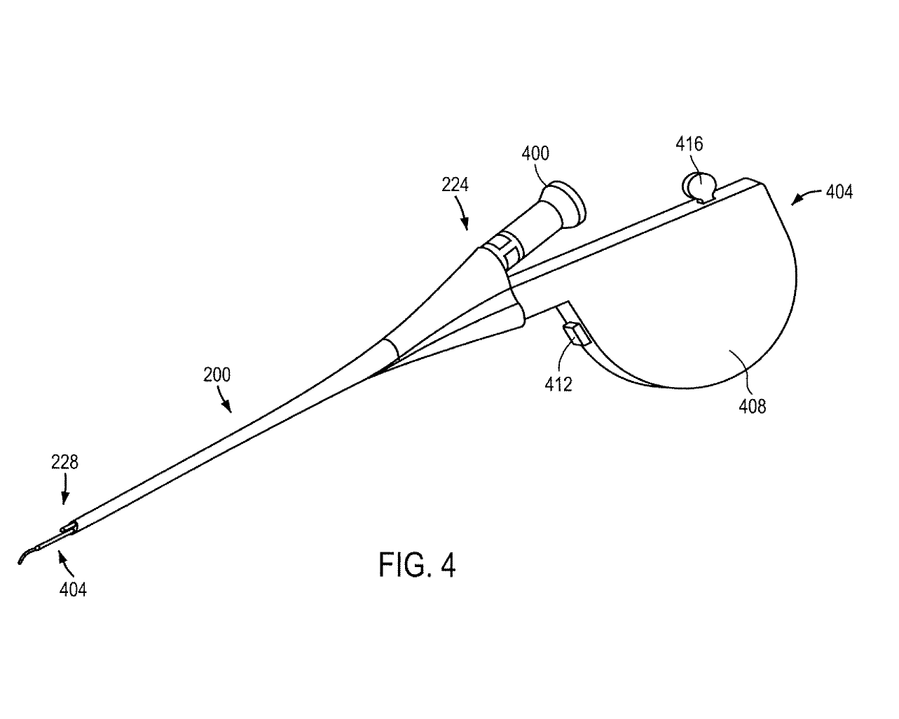

FIG. 4 illustrates the sheath 200 with a hysteroscope 400 positioned in the first lumen 208 of the sheath 200 and a device delivery system 404 positioned in the second lumen 212 of the sheath 200. The proximal end of the device delivery system 404 extends out the proximal end of the sheath 200 and, in a deployed position, the distal end of the device delivery system 404 extends out the distal end of the sheath 200, the distal end of the device delivery system 404 including the contraceptive device.

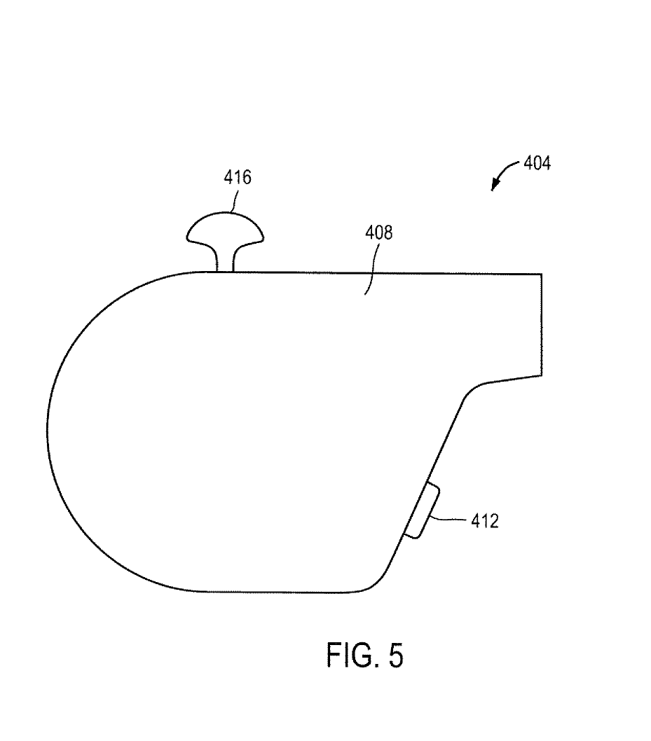

As shown in FIGS. 4 and 5, the device delivery system 404 includes a handle 408 having a first actuator 412 and a second actuator 416. In FIG. 4, the first actuator 412 is a push button actuator and the second actuator 416 is a toggle or trigger actuator. It will be appreciated that other actuators may be used for first actuator 412 and/or second actuator 416, such as, for example, a push button, a toggle switch, a trigger, a tab slider, a thumb wheel and the like. In one embodiment, the handle 408 includes an auxiliary actuator, such as a thumb wheel, if one or both of the first actuators 412, 416 fail. The handle 408 is configured to fit comfortably in the operator's hand. In one embodiment, the operator is able to hold the handle 408 and operate both the first actuator 412 and second actuator 416 with one hand.

The device delivery system 404 may be interlockable with the second lumen 212 of the sheath 200. For example, the device delivery system 404 may include a groove (not shown) to engage with the tab 232 in the second lumen 208.

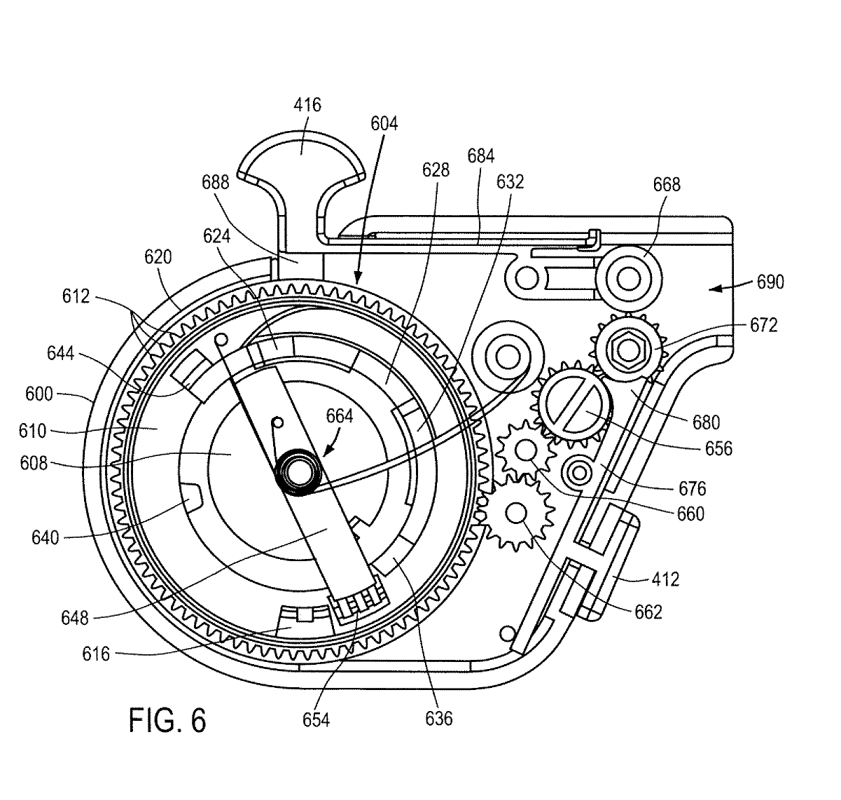

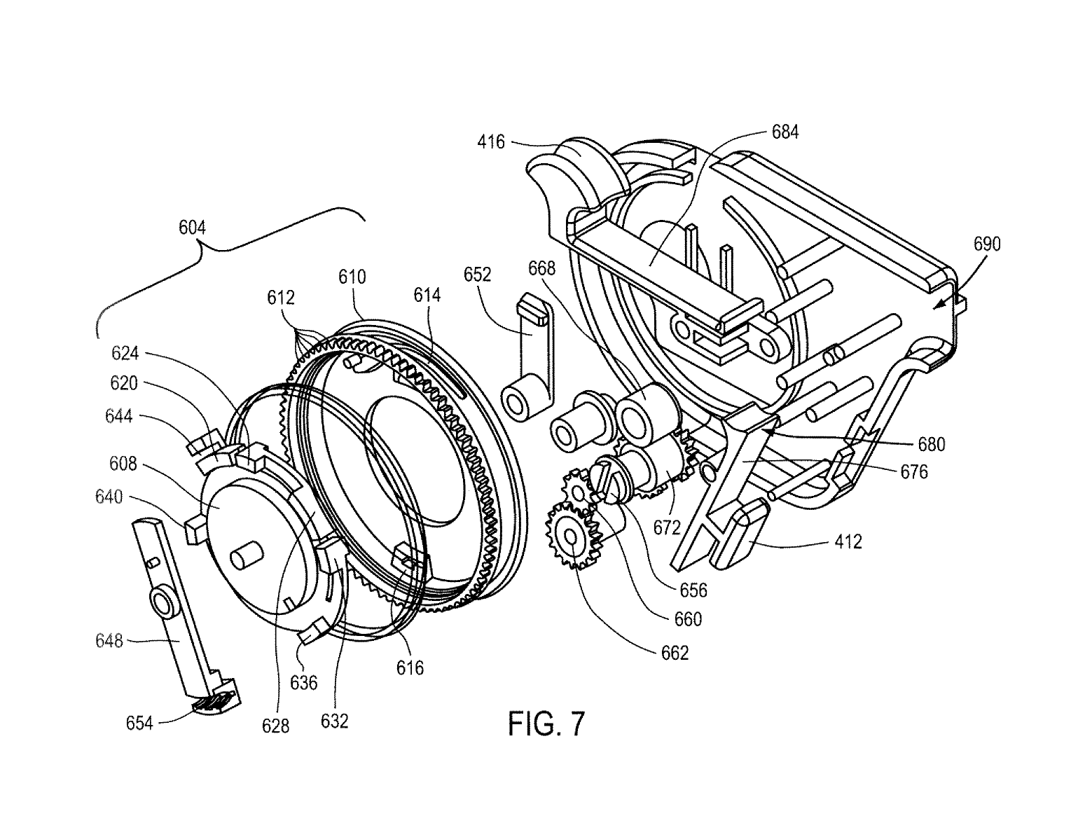

As shown in FIGS. 6 and 7, the handle 408 of the device delivery system 404 includes a housing 600. The housing 600 includes a spool 604 that includes an inner drum 608 and an outer drum 610. The outer drum 610 includes a plurality of teeth 612, an opening 614 and a projection 616. The inner drum includes six projections: a first projection 620, a second projection 624, a third projection 628, a fourth projection 632, a fifth projection 636 and a sixth projection 640. The first projection 620 includes a groove 644. The housing 600 also includes an outer sheath arm 648 and a release catheter arm 652. The outer sheath arm 648 includes a groove 654. The housing 600 further includes an opening 690. In one embodiment, the housing 600 includes a pierceable or removable membrane (not shown) to maintain a sterile environment within the housing 600.

The inner drum 608 sits within the outer drum 610 and shares the same axis of rotation as the outer drum 610. An outer sheath (not shown) of the catheter 420 is wound around the inner drum 608 through the opening 614 in the outer drum 610, between the feed rollers 668, 672 to approximately the opening 690 in the housing 600. The outer sheath of the catheter 420 is connected to the groove 654 in the outer sheath arm 648. The release catheter, which may be a delivery wire, of the catheter is secured to the inner drum 608 at groove 644.

The housing 600 also includes a first gear 656, a second gear 660, a third gear 662 and a spring motor 664. In one embodiment, the spring motor 664 includes two torsion springs, each torsion spring set in opposite directions. One end of one of the torsion springs is connected to a wall of the housing and the other end is connected to a post on the outer drum. One end of the other torsion spring is connected to a post on the inner drum and another end of the torsion spring is connected to a post on the outer drum. The main axis of both of the torsion springs is the same as the axis of rotation of the inner drum and outer drum. The torsion springs are both tensioned prior to actuation of the actuators 412, 416. The housing 600 also includes a first feed roller 668 and a second feed roller 672. The first actuator 412 is connected to a first rack 676 that has a curved end 680. The second actuator 416 is connected to a second rack 684 that includes a connector 688. The connector 688 is connected with the release catheter arm 652.

The curved end 680 of the first rack 676 is connected to the second feed roller 672 and first gear 656 such that the spring motor 664 cannot rotate the outer drum 610, until the first actuator 412 is actuated. For example, as shown in FIG. 6, the curved end 680 is wedged between the roller and gear such that neither the feed roller nor the gear can rotate. When the first actuator 412 is actuated, the curved end 680 of the first rack 676 is deflected such that the first gear 656 and feed roller 672 can rotate, releasing tension on the torsion spring connected to the outer spring. The first gear 656 is engaged with the second gear 660 and the feed roller 672, which is engaged with the third gear 662, which, in turn, is engaged with the plurality of teeth 612 of the outer drum 610. Thus, rotation of the outer drum 610 results in rotation of the feed roller 672. Rotation of the outer drum 610 rotates the outer sheath arm 648 and inner drum 608 in the same rotational direction as the outer drum 610 until the outer sheath arm 648 abuts the projection 616 of the outer drum 610, as shown in FIG. 6.

The connector 688 of the rack 684 is connected to the release catheter arm 652. When the second actuator 416 is actuated, the release catheter arm 652 disengages a detent from a slot in the inner drum 608 to separate the inner drum 608 from the outer drum 610. This separation allows the inner drum 608 to rotate in an opposing direction from the rotation of the outer drum 610 by releasing tension on the other torsion spring. When the inner drum 608 is rotated in the opposite direction, the release catheter arm 652 catches on the projection 640. When the release catheter arm 652 catches the projection 640, the inner drum 608 then rotates by itself until the projection 620 catches on the projection 616.

It will be appreciated that variations to the mechanisms within the housing 600 may be different than described above. For example, one or more motors may be provided to rotate the separate drums, the actuators powering the motors. In another example, a drum is provided to unwind the catheter, while a rack system is provided to retract the outer sheath, release catheter and/or delivery wire.

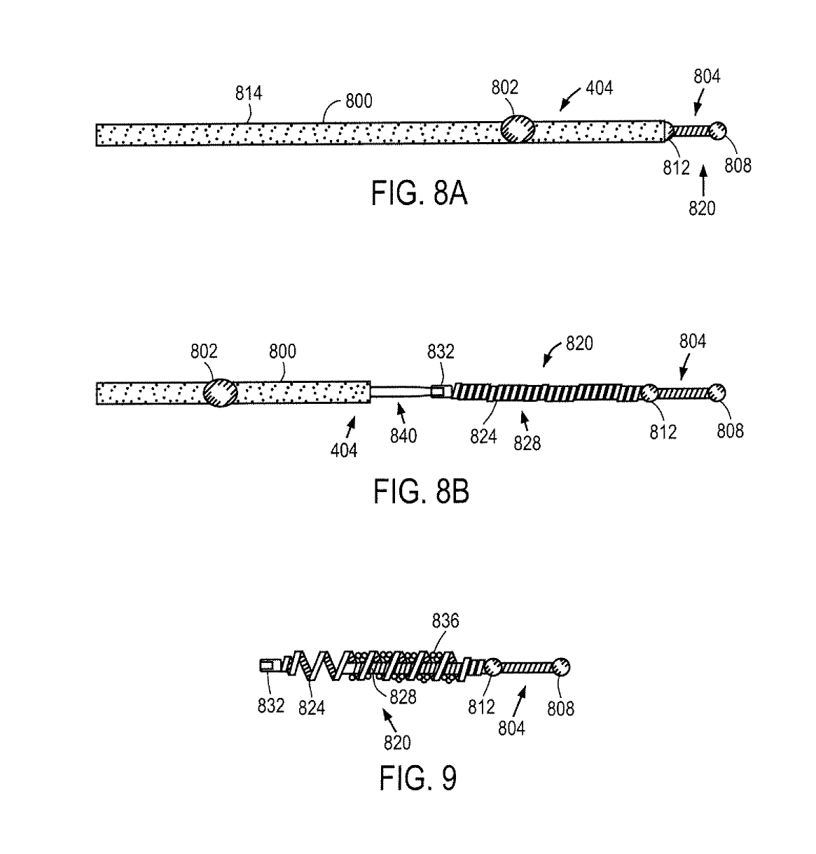

FIGS. 8A, 8B and 9 illustrate the distal end of the delivery device 404 and the implant. As shown in FIG. 8A, the delivery system 404 includes a delivery catheter 800 and a marker 802 disposed on the delivery catheter 800. The marker 802 may be one or more of the various types of conventional markers such as an optically visible marker (e.g. a marker which is colored to distinguish from its surroundings) which is visible during a hysteroscopy by visible light and a camera or a radiopaque marker or an ultrasound marker (which is visible in an ultrasound image) or other known markers which allow the user of the delivery device 404 to guide and place the distal end of the system at a proper deployment position.

In FIG. 8A, a distal portion 804 of the contraceptive device 820 is shown. The distal portion 804 includes a tip 808 and an attachment mechanism 812. The attachment mechanism 812 may be a solder bond.

FIG. 8B shows the delivery device 404 after the outer sheath 814 delivery catheter 800 has been retracted (or alternatively, the contraceptive device has been pushed relative to the delivery catheter) such that the contraceptive device 820 is fully viewable.

The contraceptive device 820 includes an outer coil 824 which is attached at attachment mechanism 812 to an inner coil 828 shown in FIG. 9. The inner coil 828 may extend from the tip 808 in a proximal direction toward an end piece 832 which is attached to the outer coil 824. The outer coil 824 is thus coupled to the inner coil 828 at attachment 812 and coupled to the end piece 832 as shown in FIG. 9.

The contraceptive device 820 shown in FIGS. 8A-8B and 9 may also include a tissue ingrowth agent 836. The tissue ingrowth agent may be a polyester fiber or other types of agents designed to cause tissue ingrowth to functionally occlude the fallopian tube. Functional occlusion of the fallopian tube is described, for example, in U.S. Pat. No. 6,526,979 which is incorporated herein by reference.

Referring back to FIG. 8B, the end piece 832 is adjacent to and abuts a release catheter 840. The release catheter 840 may include a pin or other interface designed to mate with a receptor or other interface on the end piece 832 to thereby couple the contraceptive device 820 to the release catheter 840. In one exemplary embodiment, the two interface elements on the release catheter 840 and the end piece 832 may resemble a screw and a nut which more securely secures the contraceptive device and release catheter to each other. The contraceptive device and release catheter can be released by unscrewing the contraceptive device from the release catheter after the contraceptive device has been implanted.

The contraceptive device 820 shown in FIGS. 8A-8B and 9 resembles the Essure device from Conceptus, Inc. of San Carlos, Calif., in that there is an outer coil which may be formed from a superelastic or resilient member and an inner coil which is coupled to the outer coil. The outer coil is designed to radially expand to engage the walls of a portion of the fallopian tube to thereby engage those walls and hold the device within the fallopian tube.

It will be appreciated that other contraceptive devices and/or delivery device configurations may be used. For example, the contraceptive devices described in copending U.S. application Ser. No. 10/866,493, filed Jun. 10, 2004, entitled Medical Devices and Methods of Making and Using Such Medical Devices, the entirety of which is hereby incorporated by reference, may be used as an alternative to the contraceptive device 820 described above. Other exemplary contraceptive devices include spider-like, stent-like, coil-like, or other implantable contraceptive devices.

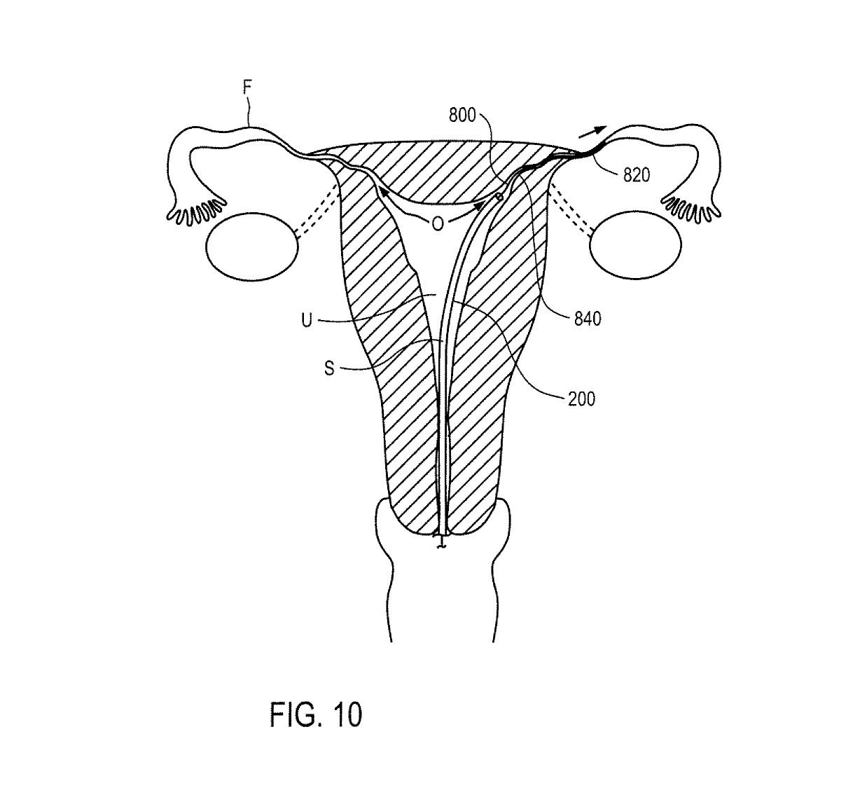

FIG. 10 illustrates placement of the contraceptive device 820 in the ovarian pathway with the delivery sheath 200. As shown in FIG. 10, the device 820 may extend proximally beyond ostium O into uterus U by a distance of, for example, about 0.2 to 1.2 cm. The device 820 may also extend distally beyond the intermural section INT and/or uterotubal junction by a distance of, for example, at least about 0.6 cm. It will be appreciated that the values provided above are exemplary and that the values may vary. In addition, it will be appreciated that there may be no proximal extension, no distal extension or neither proximal nor distal extension of the contraceptive device 820.

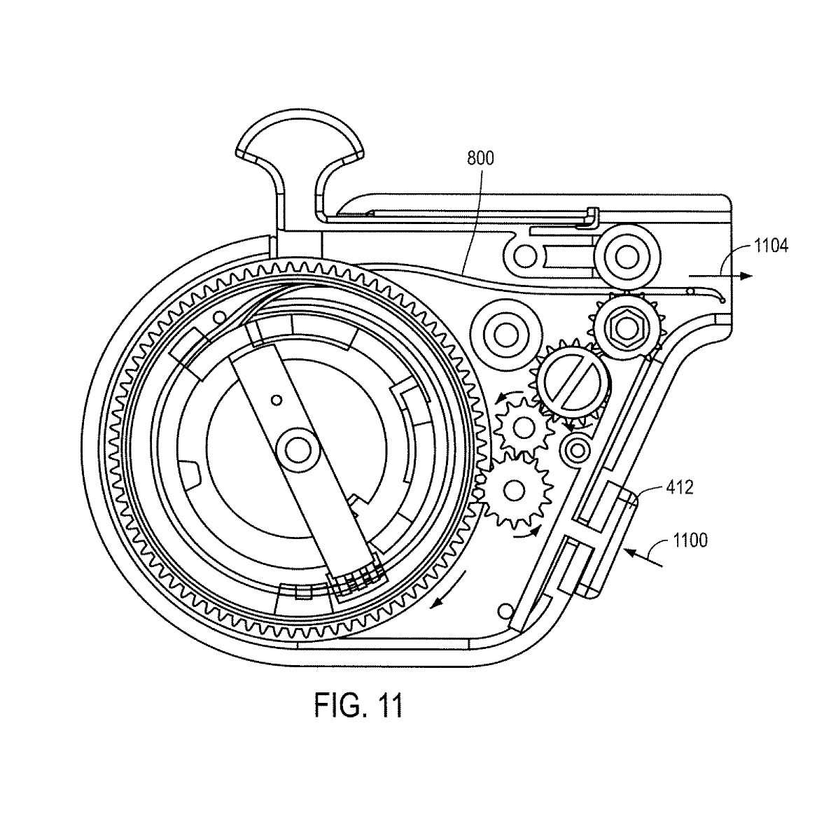

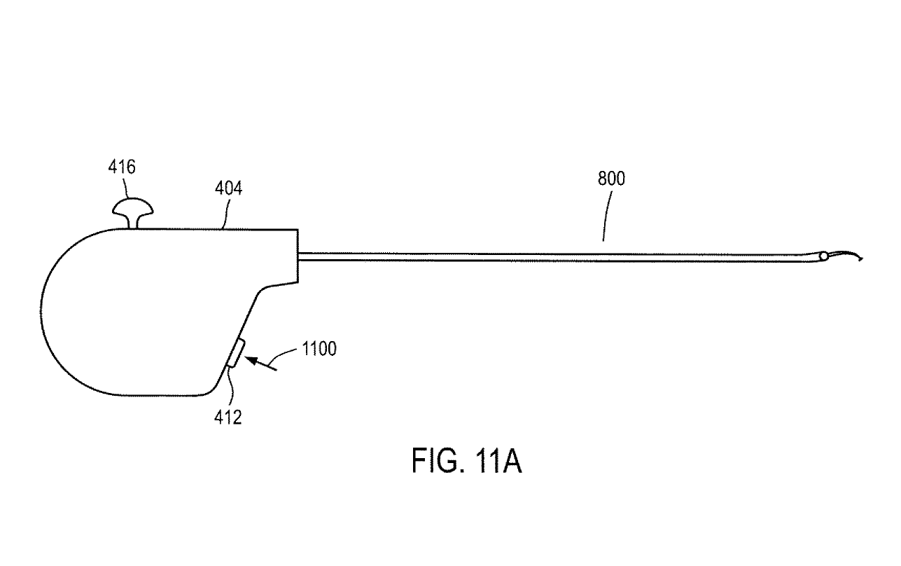

As shown in FIG. 11, when the first actuator 412 is pressed downward (arrow 1100), the spring motor causes rotation of the feed roller, gears, and both inner drum and outer drum. Rotation of the drums causes substantially the entire catheter to unwind and exit the housing (arrow 1104) between the feed rollers and through the opening of the housing. FIG. 11A illustrates the catheter in the deployed position after actuation of the first actuator 412. The operator may need to press the first actuator 412 until substantially the entire catheter exits the housing (e.g., 2-30 seconds).

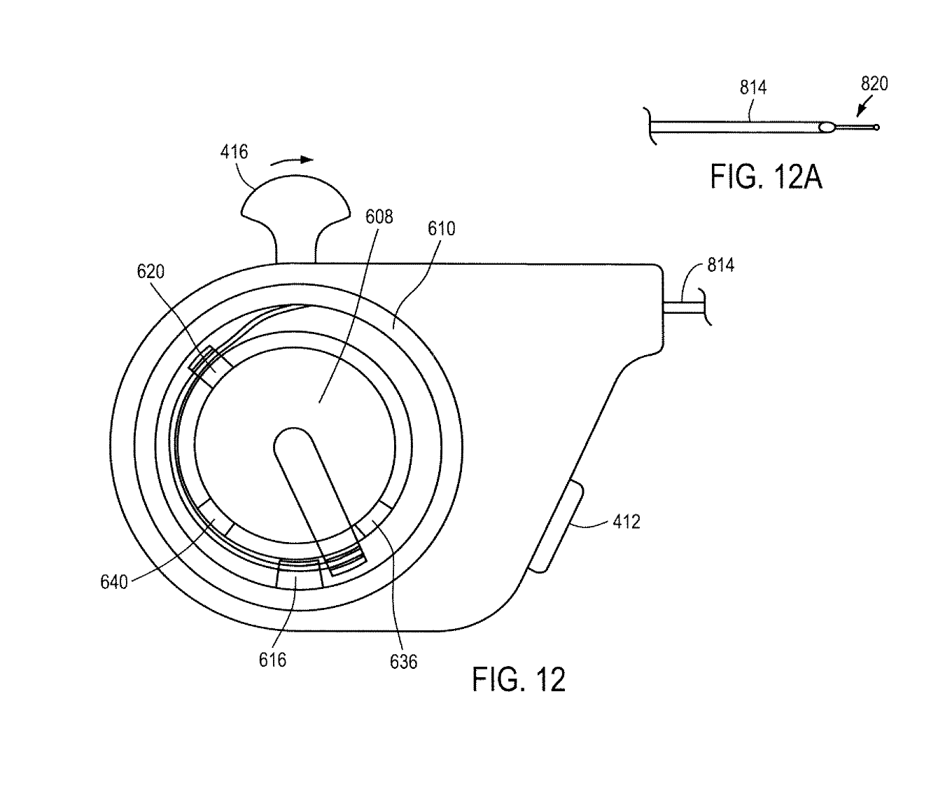

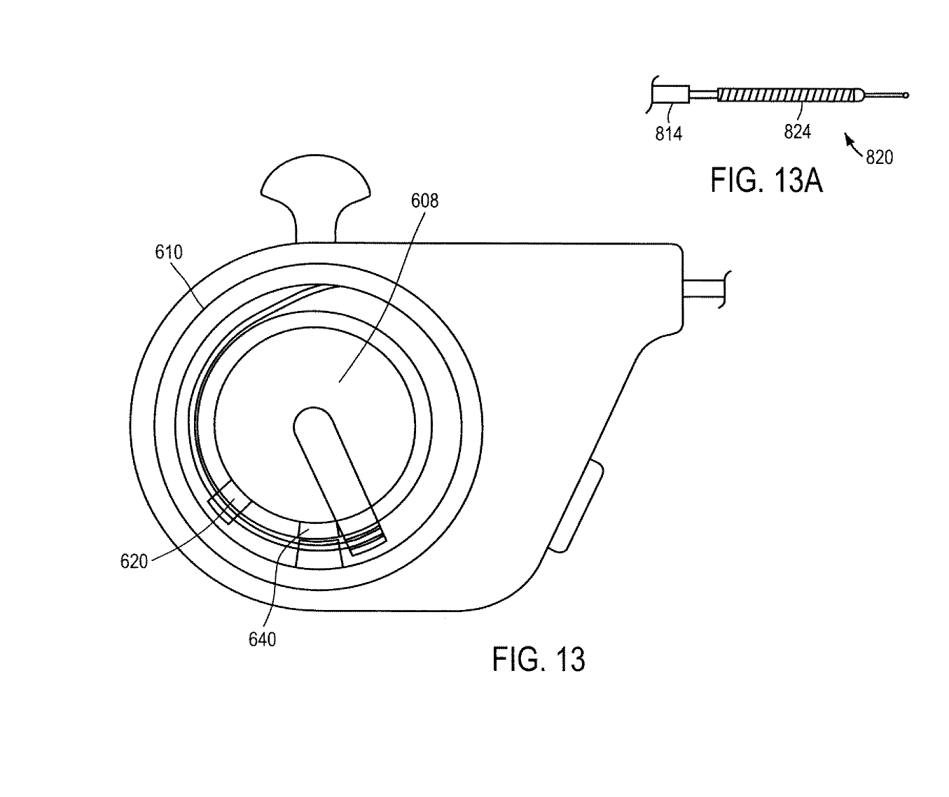

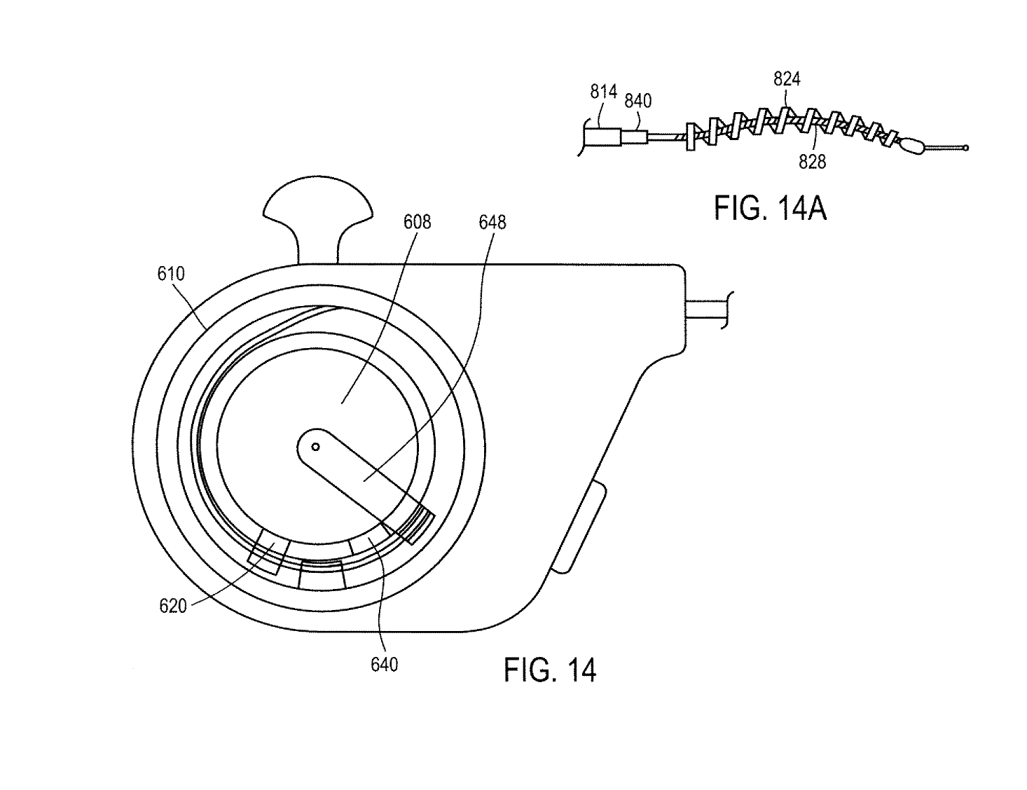

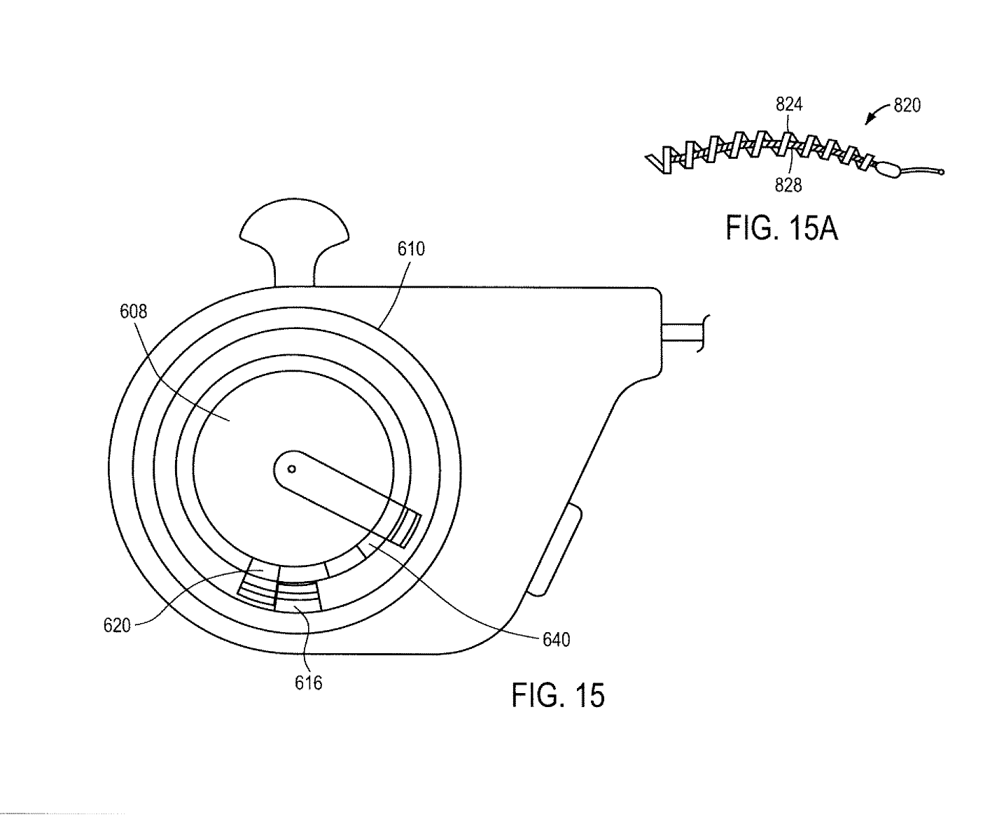

FIGS. 12-15 illustrate the result of actuating the second actuator 416. As shown in FIG. 12, the inner drum 608 rotates in an opposite direction from the rotation of the outer drum 610 to withdraw the outer sheath of the catheter, as shown in FIG. 12A. The inner drum 610 rotates until the release catheter arm 652 catches on the projection 640, as shown in FIG. 13, to fully expose the contraceptive device 820, as shown in FIG. 13A. When the release catheter arm 652 catches the projection 640, as shown in FIG. 14, the inner drum 608 then rotates by itself to expand the contraceptive device 820, as shown in FIG. 14. The inner drum continues to rotate until the projection 620 catches on the projection 616, as shown in FIG. 15, to release the delivery wire from the implant, as shown in FIG. 15A.

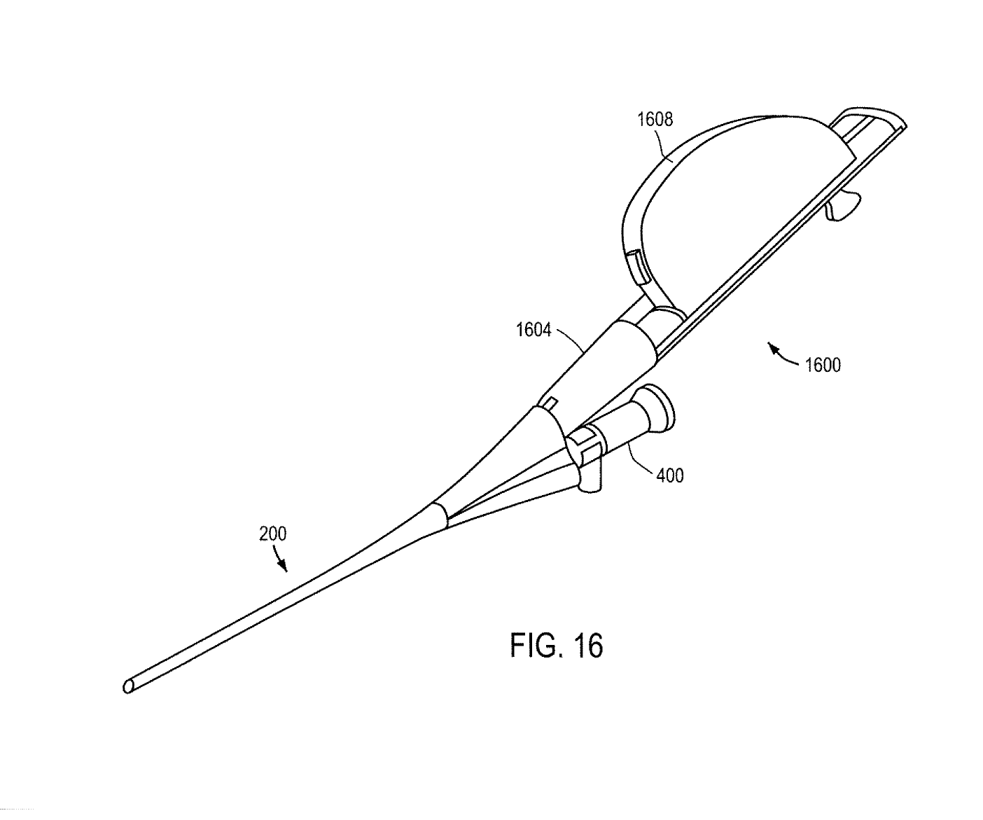

FIG. 16 illustrates the sheath 200 with the hysteroscope 400 positioned in the first lumen 208 of the sheath 200. FIG. 16 also shows a delivery system 1600 positioned in the second lumen 212 of the sheath 200. The delivery system 1600 includes a guide 1604 and a delivery device system 1608. The delivery device system 1608 is the delivery device system 404 described above with reference to FIGS. 4-15A, as described hereinabove. The delivery device system 1608 is slideably coupled to the guide 1604. The guide 1604 is secured to the sheath 200.

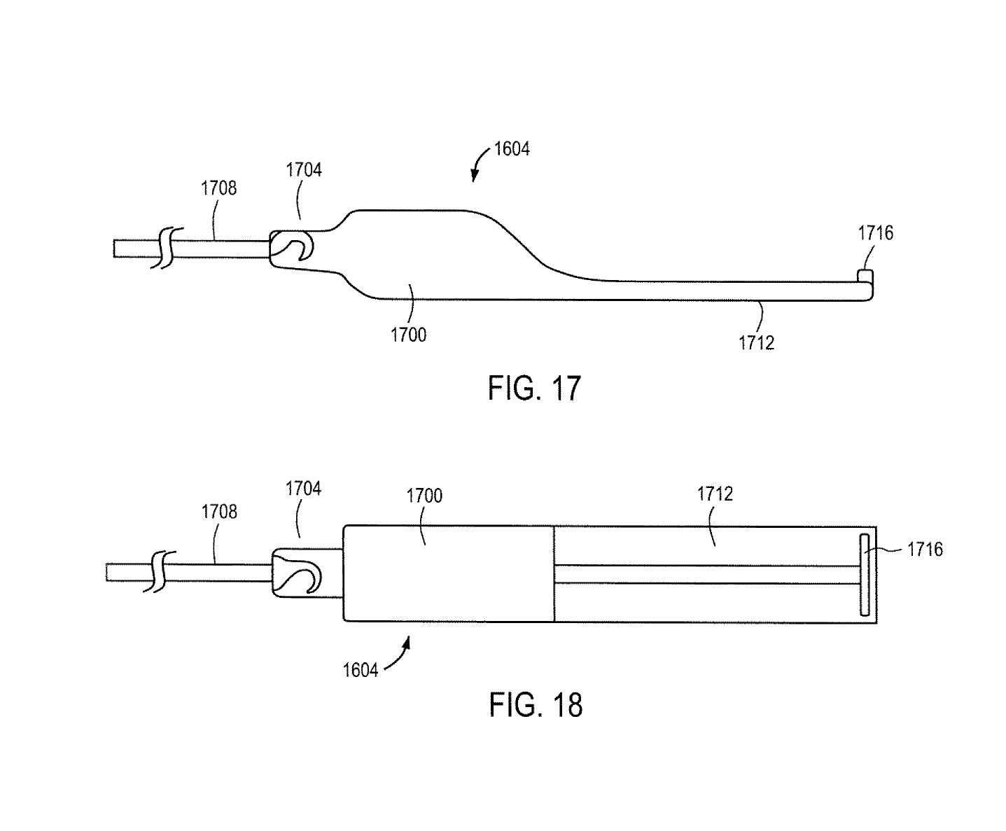

FIGS. 17-18 illustrates the guide 1604 in further detail. As described above, the guide 1604 is secured to the sheath 200, which allows an operator to manipulate the delivery device system 1608 with one hand. The guide 1604 also helps protect the delivery device system 1608 from damage.

The guide 1604 includes a body 1700, an interlock 1704, a sheath 1708, a rail 1712 and a stopper 1716. The delivery device system 1608 is coupled to the guide 1604, such that the handle of the delivery device system 1608 can slide along the rail 1712.

The interlock 1704 has a diameter that is smaller than the body 1700 and is configured to be inserted into the second lumen 212 of the sheath 200. The shape and size of the interlock 1704 is complementary to the shape and size of the second lumen 212 of the sheath. The interlock 1704 includes a groove 1720. The groove 1720 is configured to engage with the tab 232 in the second lumen 212 to lock the guide 1604 to the sheath 200.

The hard sheath 1708 is configured to protect the outer sheath of the delivery device system 1608 from damage. The sheath 1708 is configured to be inserted into the second lumen 212 at the proximal end and extends to the distal end 228 of the sheath 200. In one embodiment, the distal end of the hard sheath 1708 is aligned with the distal end 228 of the sheath 200 (i.e., the distal end of the hard sheath 1708 is flush with the distal end 288 of the delivery sheath 200). Alternatively, a portion of the hard sheath 1708 may extend beyond the distal end 228 of the sheath 200.

An exemplary length of the sheath 1708 is about 29-30 cm. It will be appreciated that the length of the sheath 1708 may be less than 29 cm or greater than 30 cm. In one embodiment, the sheath 1708 includes a distension valve to prevent fluid leakage through the sheath 1708.

An exemplary material for the sheath 1708 is stainless steel. It will be appreciated that the sheath 1708 can be formed from other materials, such as, for example, other metals, hard plastics or composites.

The rail 1712 is configured to allow the handle of the delivery device system 1608 to slide along the rail 1712. Thus, the rail 1712 is a slider assembly. The shape of the rail 1712 is generally complementary to the shape of the handle of the delivery device system 1608. For example, if the handle is curved, the rail 1712 may be also be curved, the curve of the rail 1712 having a diameter that is slightly larger than the diameter of the handle. In another example, if the handle is rectangular, the rail 1712 may also be rectangular, the width of the rail being slightly larger than the width of the handle.

The rail 1712 extends distally away from the body 1700. The stopper 1716 is provided at a distal end of the rail 1712 to limit movement of the handle relative to the body 1700. In one embodiment, the length of the rail 1712 is any value or range of values between about 1 and 10 cm in length to allow for about 1-10 cm of corresponding movement of the handle. Thus, the rail 1712 both allows longitudinal movement of the handle but also limits its longitudinal movement.

The rail 1712 is sufficiently deep to allow for movement of the handle without the handle coming off the rail 1712, but sufficiently shallow to allow for removal of the handle from the rail 1712. The rail 1712 may have openings (not shown) to allow an operator to more easily remove the handle from the rail 1712. In addition, the rail 1712 may have a track (not shown) to improve movement along the rail 1712.

The guide may also allow for rotational movement of the rail relative to the delivery sheath 200. For example, the body of the guide may include a ball and socket joint.

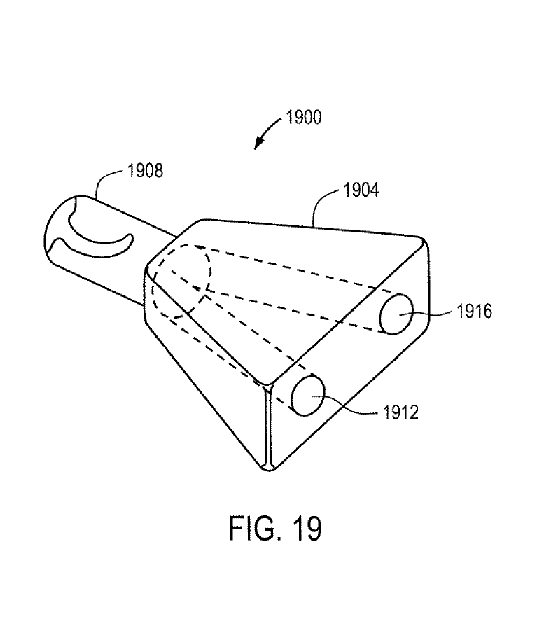

FIG. 19 illustrates an adapter 1900 that may be inserted into the second lumen 212 of the sheath 200. The adapter 1900 is configured to receive two device delivery systems 404, as described above with reference to FIG. 4, or is configured to receive two delivery systems 1600, as described above with reference to FIG. 16. The adapter 1900 includes a body 1904, an interlocking element 1908, a first opening 1912 and a second opening 1916. The interlocking element 1908 is configured to engage with a corresponding interlocking element in the second lumen 212 of the sheath 200. The first opening 1912 and second opening 1916 may each include an interlocking feature (not shown) to secure the device delivery systems or delivery systems to the adapter 1900.

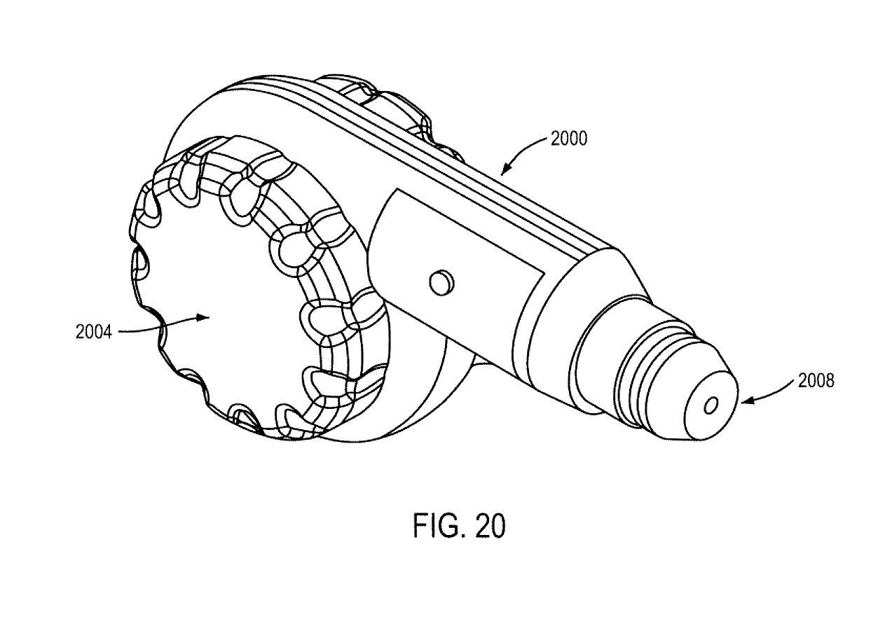

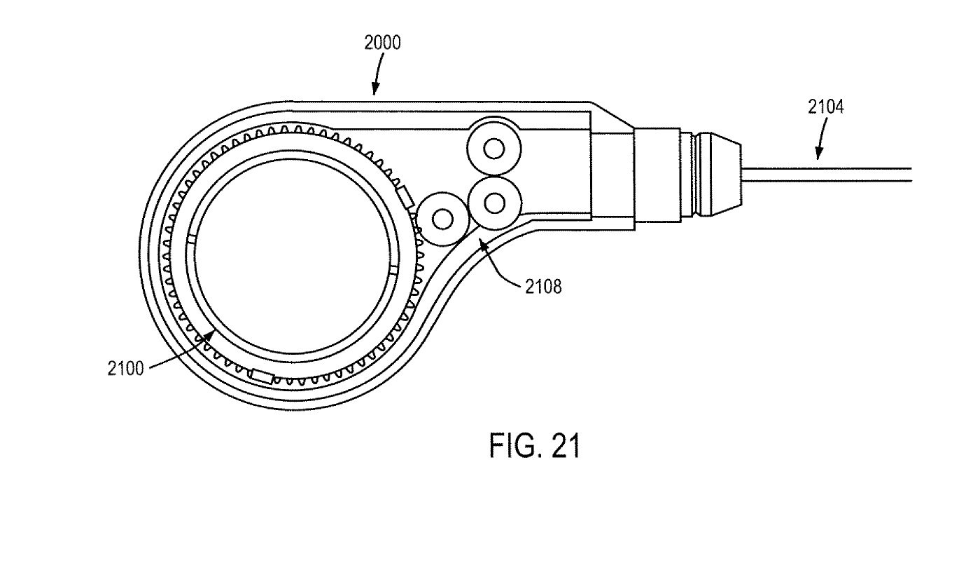

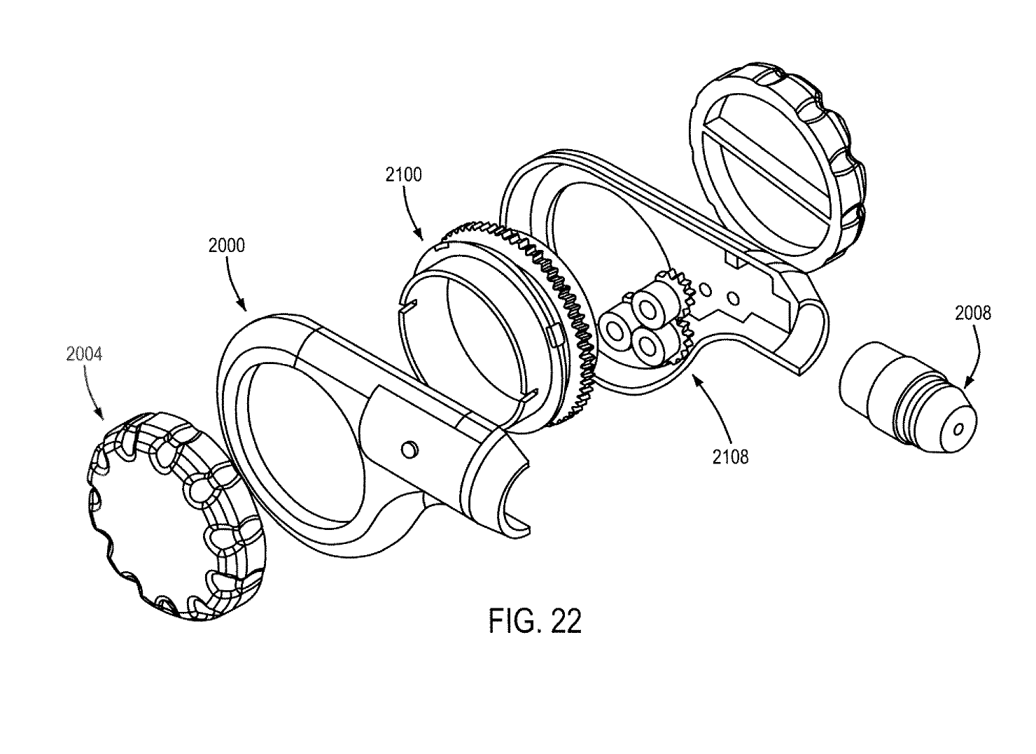

FIGS. 20 through 23 illustrate an alternative embodiment of the implant delivery device. The housing 2000, circular grip 2004 and opening for the catheters are shown in FIG. 20. A cross sectional view of the housing interior is shown in FIG. 21, with reference to the spool 2100 and gears and feed rollers 2108. The delivery catheter 2104 is also shown connected to the opening. An exploded view of the device is provided in FIG. 22.

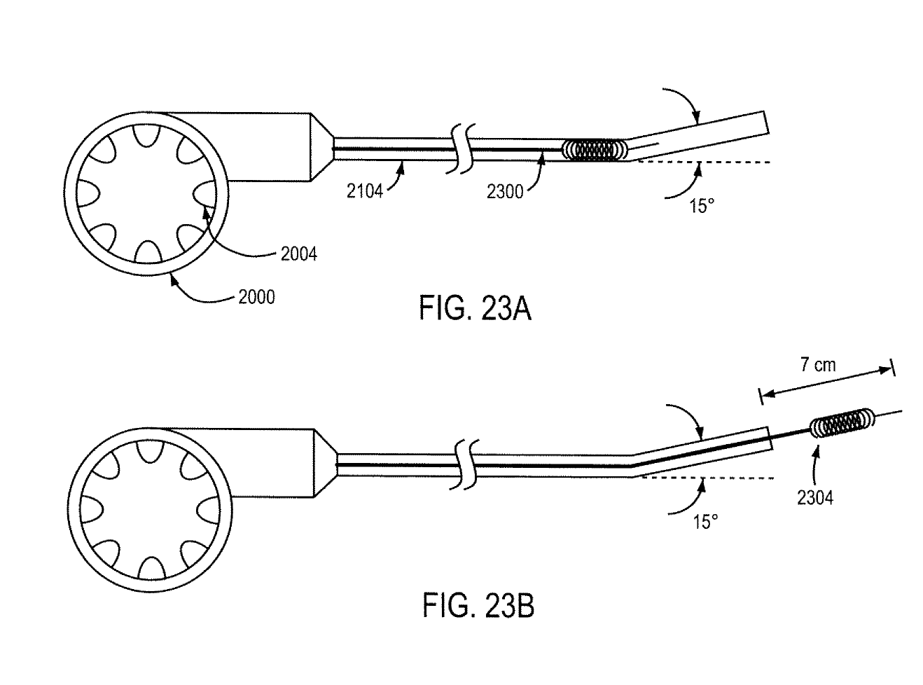

A cross sectional view of the entire delivery device is shown in FIGS. 23A and 23B. The distal end of the delivery catheter 2104 is bent at a 15 degree angle from its longitudinal axis. The operator can align this bend with curves encountered in the ovarian pathway by manually rotating the housing. This facilitates the navigation of the delivery catheter through the numerous twists and turns in the ovarian pathway.

In FIG. 23A the release catheter 2300 and expandable implant 2304 are shown in a retracted position within the delivery catheter 2104. As the circular grip 2004 is rotated clockwise, the release catheter is unwound from the spool 2100 and advanced forward along with the expandable implant 2304 through the length of the delivery catheter 2104 until it reaches the fully deployed position as shown in FIG. 23B. Upon reaching the fully deployed position, the distal end of the expandable implant protrudes at least 7 cm beyond the distal end of the delivery catheter. This advancing motion capability allows the operator to deliver the contraceptive device (expandable implant) to the desired location in the ovarian pathway. The release catheter can likewise be retracted by a counterclockwise rotation of the circular grip.

When the expandable implant 2304 is correctly positioned within the ovarian pathway it is separated from the release catheter by any one of a number of means such as a trigger mechanism (not shown), a manual rotation of the release catheter, or an automatic release caused by a quick pullback of the release catheter.

The foregoing description with attached drawings is only illustrative of possible embodiments of the described method and should only be construed as such. Other persons of ordinary skill in the art will realize that many other specific embodiments are possible that fall within the scope and spirit of the present idea. The scope of the invention is indicated by the following claims rather than by the foregoing description. Any and all modifications which come within the meaning and range of equivalency of the following claims are to be considered within their scope.

1. An implant delivery device comprising:
a housing;
an expandable implant;
a release catheter, the expandable implant releasably coupled with the release catheter;
a delivery catheter, the release catheter and the expandable implant slideable through the delivery catheter;
a spool in the housing, at least a portion of the delivery catheter wound around the spool in a first position;
a first actuator coupled with the housing and the spool to rotate the spool to unwind the delivery catheter from the spool in a second position; and
a second actuator coupled with the housing and the spool to release the implant from the release catheter.

2. The implant delivery device of claim 1, wherein the release catheter comprises a wire, the implant releasably coupled to the wire.

3. The implant delivery device of claim 1, wherein the first actuator comprises a push button and the second actuator comprises a toggle or trigger.

4. The implant delivery device of claim 1, wherein the spool comprises an inner drum and an outer drum.

5. The implant delivery device of claim 1, further comprising a spring motor coupled with the spool.

6. The implant delivery device of claim 5, further comprising a plurality of gears and feed rollers coupled with the spring motor, spool and first actuator.

7. The implant delivery device of claim 4, wherein the first actuator is coupled with the outer drum and the second actuator is coupled with the inner drum.

8. The implant delivery device of claim 1, wherein the housing comprises an opening, the delivery catheter to exit the housing through the opening when the first actuator is actuated.

9. The implant delivery device of claim 8, further comprising a pierceable seal over the opening.

10. A method for delivering a contraceptive device to an ovarian pathway comprising:
in response to actuation of a first actuator, unwinding a catheter wound around a spool in a housing to deliver the contraceptive device near the ovarian pathway; and
in response to actuation of a second actuator, releasing the contraceptive device from the catheter.

11. The method of claim 10, wherein releasing the contraceptive device from the catheter further comprises withdrawing an outer sheath of the catheter.

12. The method of claim 10, wherein releasing the contraceptive device from the catheter further comprises expanding the contraceptive device.

13. The method of claim 10, wherein delivering the contraceptive device near the ovarian pathway comprises delivering the contraceptive device to the ovarian pathway.

14. The method of claim 10, wherein unwinding a catheter wound around a spool in a housing comprises rotating an outer drum of the spool with a spring motor.

15. An implant delivery device comprising:
a housing;
an expandable implant;
a release catheter, said expandable implant releasably coupled with said release catheter;
a delivery catheter, said release catheter and said expandable implant slideable through said delivery catheter;
a spool in said housing, at least a portion of said release catheter wound around said spool; and
a circular grip coupled with said housing and said spool to rotate said spool to unwind said release catheter from said spool and advance said expandable implant through said delivery catheter.

16. The implant delivery device of claim 15, wherein the distal end of said delivery catheter is bent at an angle of approximately 15 degrees from the longitudinal axis of said delivery catheter, said bend occurring at the point coincident with the distal end of said expandable implant.

17. The implant delivery device of claim 15, wherein said release catheter comprises a wire, said expandable implant releasably coupled to said wire.

18. The implant delivery device of claim 15, further comprising a plurality of gears and feed rollers coupled with said circular grip and said spool to regulate the unwind rate of said release catheter from said spool.

19. The implant delivery device of claim 15, wherein said housing comprises an opening, said release catheter to advance forward exiting said housing through said opening when said circular grip is rotated clockwise.

20. The implant delivery device of claim 15, wherein said release catheter retracts backward into said housing through said opening when said circular grip is rotated counterclockwise.

21. The implant delivery device of claim 15, wherein the distal end of said expandable implant protrudes at least approximately 7 cm from the distal end of said delivery catheter as said release catheter is advanced forward by the clockwise rotation of said circular grip.

22. The implant delivery device of claim 15, further comprising a pierceable seal over said opening.

23. A method for delivering a contraceptive device to an ovarian pathway comprising:
rotating a housing about the longitudinal axis of a delivery catheter to allow a bend in the distal end of said delivery catheter to align with bends in said ovarian pathway thereby easing the advancement of said delivery catheter through said ovarian pathway;
rotating a circular grip in a clockwise direction to unwind a release catheter wound around a spool in said housing to advance said contraceptive device toward a desired location in said ovarian pathway; and
releasing said contraceptive device from said release catheter.

24. The method of claim 23, wherein releasing said contraceptive device from said release catheter further comprises one of actuating a release trigger, manual rotation of said release catheter, automatic release on pull back of said release catheter or any other known means of releasing.

25. The method of claim 23, wherein releasing said contraceptive device from said release catheter further comprises expanding said contraceptive device.
